(19) United States
(12) Patent Application Publication
(10) Pub. No.: US 2011/0237836 A1
(43) Pub. Date: Sep. 29, 2011

(54) Method for Producing Alkylnitrobenzenes and Alkylanilines, Unbranched in the 1'-Position, from Nitrotoluenes

(75) Inventor: Alexander Straub, Wuppertal (DE)

(73) Assignee: Bayer CropScience AG, Monheim (DE)

(21) Appl. No.: 12/373,164

(22) PCT Filed: Jul. 12, 2007
(86) PCT No.: PCT/EP07/06176

§ 371 Date: Nov. 5, 2009

(30) Foreign Application Priority Data
Jul. 14, 2006 (DE) 10 2006 033 091.9

(51) Int. Cl. C07C 209/36; C07C 205/06; C07C 201/12

(52) U.S. Cl. 564/420; 568/940; 568/928

(57) ABSTRACT

The present invention relates to a process for preparing nitrobenzene derivatives and aniline derivatives, which are of significance as intermediates for fungicidally active alkylanilides.

The present invention relates to a process for preparing nitrobenzene derivatives and aniline derivatives, which are of significance as intermediates for fungicidally active alkylanilides.

The prior art already describes preparation methods for 1′-unbranched alkylanilines. These include the Friedel-Crafts acylation of anilines with acid chlorides and subsequent reduction of the resulting ketones (EP-A-824099) and the palladium- or copper-catalyzed reaction of bromoalkylbenzenes with benzophenone imine or ammonia, if appropriate followed by the elimination of the protecting group with hydroxylamine (WO-A-03074491 and WO-A-06061226).

Alkylnitrobenzenes can be converted to alkylanilines by reducing the nitro group and have been obtained to date, for example, by the nitration of alkylaromatics (EP-A-824099; WO-A-03074491) or the reaction of nitrobenzene derivatives with Grignard reagents (J. Org. Chem. 1980, 45, 522).

Nitro groups can, however, lead to various redox by-products in Grignard reactions.

J. Organomet. Chem. (2006), 691(8), 1462 describes the synthesis of 1-[3,3-dimethylbut-1-en-1-yl]-2-nitrobenzene proceeding from 2-nitrobenzoyl chloride. Owing to the high costs and the toxicity of the reagents used there, for example Me₃SnF, polymethylhydroxysiloxane and Pd₂(dba)₃, the method cannot be practiced in an economically viable manner in an industrial process.

Alkenylnitrobenzenes, for example 1-(2-nitrophenyl)-1,3-butadiene, have to date been obtainable only by the complicated route shown in scheme (I) (cf. U.S. Pat. No. 2,626,960).

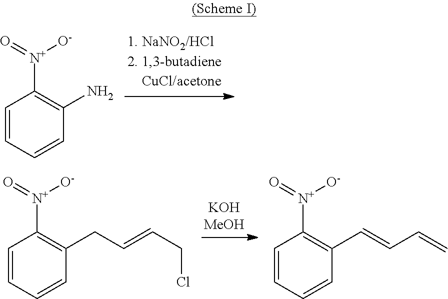

Heck reactions of 2-halonitroaromatics with alkenes have likewise been described before in the prior art (Synthesis 2005,2193; Adv. Synth. Catal. 2002, 344, 172). In this reaction, chloroaromatics are generally significantly less reactive than bromo- or iodoaromatics. The reaction of 2-bromonitrobenzene with vinylboronic acid, for example, leads to a yield of 74% 2-vinylaniline, while no yield at all is obtained with 2-chloronitrobenzene (JOC 2002, 67, 4968). Specifically for an economically viable production process, however, only chloroaromatics are an option.

In the case of ortho-substituted compounds, furthermore, additional inhibition of reaction is found.

The processes described are therefore unselective, complex and/or uneconomic.

It is thus an object of the present invention to provide a process for preparing 1′-unbranched alkyl- and/or alkenylanilines and -nitrobenzenes. In contrast to the processes described in the prior art, the 1′-unbranched alkylanilines should be obtainable with improved selectivities and in high purities and yields.

The object is surprisingly achieved by a process for preparing nitrobenzene derivatives, especially alkenylnitrobenzenes, of the formula (I)

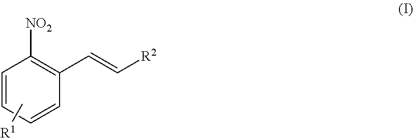

where R¹ is hydrogen, halogen, —CR′(CF₃)₂ where R′ is selected from H, F and an O—C₁₋₄-alkyl group and is preferably hydrogen, the R¹ substituent is preferably in the meta or para position, more preferably in the 4 position (para to the NO₂ group) of the aromatic
and R² is i-propyl, cyclopropyl, ethylenyl or t-butyl, characterized in that 2-halonitrobenzenes of the formula (II)

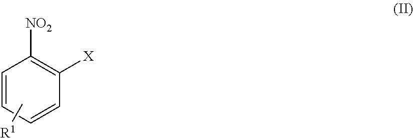

where
R¹ is as defined above and
X is a halogen atom, preferably Cl or Br, more preferably Cl,
are coupled with alkenes of the formula (III)

R²—CH═CH₂ (III)

where R² is as defined above.

A second embodiment of the invention relates to nitrobenzene derivatives of the general formula (IV)

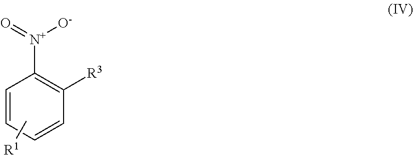

where
R¹=hydrogen, halogen, —CR′(CF₃)₂ where R′ is selected from H, F and —O—C₁₋₄-alkyl; and
R³=—CH═CH-i-Prop, —CH₂CH₂-t-But, —CH₂CH₂-i-Prop and

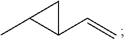

or
R¹=halogen, —CR′(CF₃)₂ where R′ is selected from with R′═H, F and —O—C₁₋₄-alkyl; and

### R³=—CH═CH-t-But,

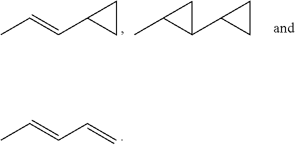

The process according to the invention can be illustrated by way of example by the following scheme (II):

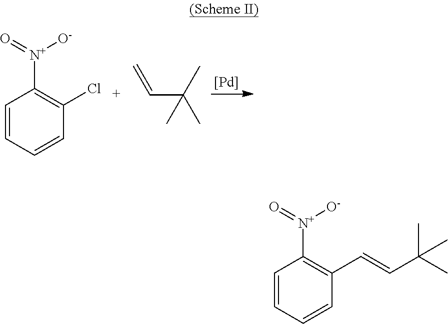

The 1-[3,3-dimethylbut-1-en-1-yl]-2-nitrobenzene which results according to scheme (II) can advantageously be converted by hydrogenation in one step to 2-(3,3-dimethyl-butyl)phenylamine, which is described in WO-A-05042494 as an intermediate for active agrochemical ingredients.

The synthesis known to date proceeds, however, via a complicated Sonogashira reaction of the expensive 2-bromoacetanilide with the expensive dimethylbutyne, subsequent hydrogenation and deacetylation, and is therefore complicated and uneconomic.

The process according to the invention can also be illustrated by the following advantageous example according to scheme (III):

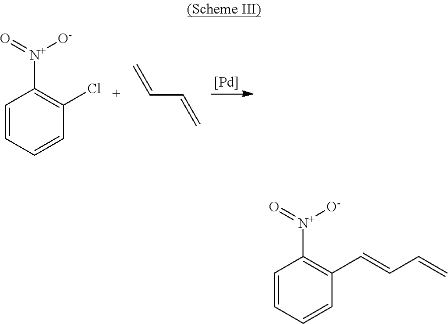

In connection with the present invention, the term “halogens” encompasses elements which are selected from the group consisting of fluorine, chlorine, bromine and iodine, preference being given to using fluorine, chlorine and bromine and particular preference to using chlorine and bromine.

Optionally substituted radicals may be mono- or polysubstituted, and the substituents may be the same or different in the case of polysubstitutions.

The definition C₁-C₄-alkyl encompasses the largest range for an alkyl group defined herein. Specifically, this definition encompasses the meanings of methyl, ethyl, n-propyl, isopropyl, n-, iso-, sec- and t-butyl.

The inventive compounds may optionally be present in the form of mixtures of different possible isomeric forms, especially of stereoisomers, for example E and Z, threo and erythro, and also optical isomers, but if appropriate also of tautomers. Both the E and the Z isomers, and also the threo and erythro isomers, and also the optical isomers, any possible mixtures of these isomers, and the possible tautomeric forms are claimed.

According to the present invention, the coupling of the halonitrobenzene (II) and of the alkene (III) can be effected in the presence of a transition metal or noble metal catalyst, preferably in the presence of a palladium catalyst. Suitable catalysts are, for example, selected from the group consisting of Pd(OAc)₂, Pd(OH)₂, PdCl₂, Pd(acac)₂ (acac=acetylacetonate), Pd(NO₃)₂, Pd(dba)₂, Pd₂ dba₃ (dba=dibenzylideneacetone), dichlorobis(triphenylphosphine)palladium(II), Pd(CH₃CN)₂Cl₂, tetrakis(triphenylphosphine)palladium(0), Pd/C or palladium nanoparticles.

Based on 1 mole of the halonitrobenzene (II), the noble metal catalyst is used in a ratio of from 10.0 to 0.001 mol %, preferably from 2.0 to 0.01 mol %, more preferably from 1.0 to 0.1 mol %.

The Heck-like coupling step is performed preferably in the presence of an inorganic or organic base. Examples of organic bases are diethylamine, dipropylamine, diisopropylethylamine, di-butylamine, dicyclohexylamine, piperidine, triethylamine, tripropylamine, tributylamine, 1,8-diazabicyclo[5.4.0]undec-7-ene (DBU), 1,4-diazabicyclo[2.2.2]octane (DABCO).

Examples of inorganic bases are potassium acetate, sodium acetate, potash, soda, potassium t-butoxide, sodium t-butoxide, sodium t-amylate, preference being given to using triethylamine, tributylamine, sodium acetate and potassium acetate.

The inventive coupling step can be performed with or without addition of ligands. The ligands used may be triarylphosphines, diarylalkylphosphines, diarylphosphines, for example tri(o-tolyl)phosphine, triphenylphosphine, diphenylcycloalkylphosphines, di- and tri(cycloalkyl)-phosphines, diadamantylphosphine, dinorbornylphosphine, di-tert-butylphosphine, dicyclo-hexylphosphine, diadamantylbutylphosphine, trialkyl phosphites and BINAP (BINAP=2,2′-bis(diphenylphosphino)-1,1′-binaphthalene), dialkylphosphines, dialkylarylphosphines, trialkyl-phosphines, diaryl(dialkylamino)phosphines and arylbis(dialkylamino)phosphines and mixtures thereof, preference being given to using tri(o-tolyl)phosphine, triphenylphosphine, diphenylcycloalkylphosphines, di- and tri(cycloalkyl)phosphines, diadamantylphosphine, dinorbornylphosphine, di-tert-butylphosphine, dicyclohexylphosphine, diadamantylbutyl-phosphine, trialkyl phosphites and BINAP (BINAP=2,2′-bis(diphenylphosphino)-1,1′-binaphthalene), and particular preference being given to using tri(o-tolyl)phosphine, triphenylphosphine, diphenylmethylphosphine, diphenylneomenthylphosphine, BINAP.

In a preferred embodiment of the invention, the ligands are added to the reaction mixture in the amount needed for the desired molar ratio. The reaction mixture may comprise either a ligand-free precursor of the catalyst, for example a palladium salt such as PdCl₂ or Pd(OAc)₂, or a complex already containing the ligand, for example dichlorobis-(triphenylphosphine)palladium(II) or tetrakis(triphenylphosphine)palladium(0), to which an appropriate amount of the same or another ligand is additionally added until the desired molar ratio is established.

The inventive coupling step is preferably performed in a solvent or solvent mixture. Suitable solvents are, for example, N,N-dialkylalkanamides, for example N-methylpyrrolidone, dimethylformamide and dimethylacetamide; ketones such as acetone, diethyl ketone, methyl ethyl ketone and methyl isobutyl ketone; nitriles, for example acetonitrile and butyronitrile; ethers, for example dimethoxyethane (DME), tetrahydrofuran (THF), 2-methyl-THF and dioxane; alcohols, for example methanol, ethanol, n-propanol, isopropanol and isoamyl alcohol; water; ethylene carbonate or propylene carbonate.

In an alternative embodiment of the present invention, the coupling step is performed in the presence of water. In this case, it is possible to use triarylphosphines which are preferably substituted on the aromatic such that the water solubility of the palladium complexes formed is increased. Such substituents may, for example, be sulfonic acid radicals, carboxyl groups, phosphonic acid radicals, phosphonium groups, peralkylammonium groups, hydroxyl groups and polyether groups.

In addition, it is possible to use tetraalkonium salts such as tetrabutylammonium bromide, tetrabutylammonium acetate, aryl₄P—X (in which aryl is phenyl or o-tolyl and X is chlorine or bromine).

Examples of useful ligands also include EDTA, substituted diazabutadienes or 1,3-bis(aryl)imidazole carbenes.

The proportion of reactants relative to solvent can be varied within a wide range. The proportion of the reactants is preferably from 5 to 75% by weight, more preferably from 10 to 50% by weight, based on the mixture of solvent and reactants.

In this connection, the term “reactants” encompasses the 2-halonitrobenzenes, the alkenes, the Pd complex, the ligands and the bases.

When performing the inventive coupling step, the working temperatures are generally from 20° C. to 150° C., preferably in the range from 50° C. to 130° C.

In a preferred embodiment of the present invention, for 1 mole of the halonitrobenzene of the formula (II),

from 0.5 to 3.0 mol, preferably from 0.75 to 1.5 mol and more preferably from 1.0 to 1.2 mol of the alkene of the formula (III) and
0.00001 and 0.01 mol, preferably from 0.0001 to 0.05 mol and more preferably from 0.001 to 0.01 mol of the transition metal catalyst and
from 0.5 to 10 mol, preferably from 1 to 5 mol and more preferably from 2 to 3 mol of a base are used.

The compounds obtained by the inventive coupling can be hydrogenated by subsequent hydrogenation to compounds of the formulae (X) or (XI)

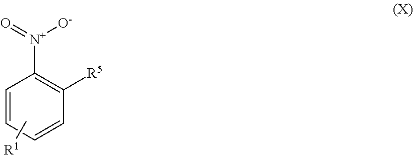

where
R¹ is hydrogen, halogen, —CR′(CF₃)₂ where R′ is selected from H, F and an O—C₁₋₄-alkyl group and is preferably hydrogen and
R⁵ is —CH₂CH₂-t-Bu, —CH₂CH₂-i-Prop, —CH₂—CH₂-cyclopropyl, and the R¹ substituent is preferably in the meta or para position, more preferably in the 4 position (para to the NO₂ group), of the aromatic;
   or

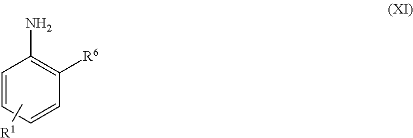

where
R¹ is hydrogen, halogen, —CR′(CF₃)₂ where R′ is selected from H, F and an O—C₁₋₄-alkyl group and is preferably hydrogen, and the R¹ substituent is preferably in the meta or para position, more preferably in the 4 position (para to the NH₂ group), of the aromatic, and
R⁶ is —CH₂CH₂-t-Bu, —CH₂CH₂-i-Prop, —CH₂—CH₂-cyclopropyl, —CH═CH-t-But, —CH═CH-i-Prop,

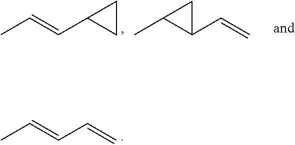

A further aspect of the present invention relates to a process for preparations of the compounds of the formula (XII)

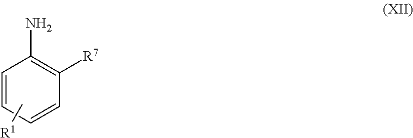

where
R¹ is hydrogen, halogen, —CR′(CF₃)₂ where R′ is selected from H, F and O—C₁₋₄-alkyl, and
R⁷ is CH₂CH₂-t-Bu, —CH₂CH₂-i-Prop,

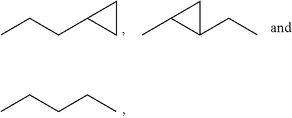

by hydrogenating compounds of the formula (XI)

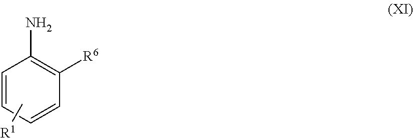

where
R¹ is hydrogen, halogen, —CR′(CF₃)₂ where R′ is selected from H, F and O—C₁₋₄-alkyl, and

### R⁶ is —CH═CH-t-Bu, —CH═CH-i-Prop,

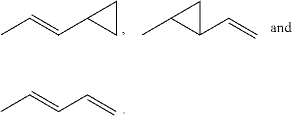

The reaction conditions of the hydrogenation are known to those skilled in the art and have been described before in the prior art, for example, in Becker, H. G. D. et al, Organikum (1976), Interdruck, Leipzig. Particular preference is given to effecting the hydrogenation in the liquid and/or gas phase in the presence of suitable hydrogenation catalysts. Suitable catalysts are especially Pd/C, PtO₂ and Raney nickel.

The hydrogenation is typically performed with hydrogen pressures of from 1 to 100 bar, preferably from 2 to 30 bar, more preferably from 5 to 10 bar and at temperatures in the range from 0 to 150° C., preferably from 10 to 100° C. and more preferably from 15 to 50° C.

Alternatively, the hydrogenation can be effected with hydrogenation reagents, which are selected, for example, from Zn, Fe, SnCl₂, Sn and dithionite.

The hydrogenation can be effected in the presence of an acid. Useful hydrogen sources also include formates and hydrazine.

A preferred example of alkylnitrobenzenes which are obtainable by the present process is that of the compounds of the following formulae (V) and (VI):

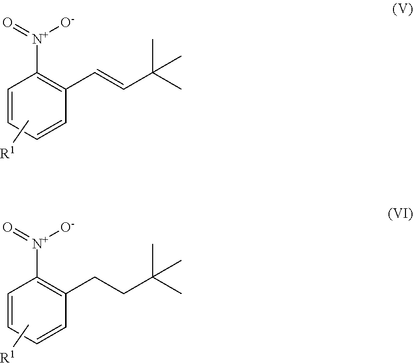

where R¹ is hydrogen, halogen, —CR′(CF₃)₂ where R′ is selected from H, F and O—C₁₋₄-alkyl and is preferably hydrogen, and the R¹ substituent is preferably in the meta or para position, more preferably in the 4 position (para to the NO₂ group) of the aromatic.

In a further embodiment of the process according to the invention, the compounds of the formula (XII) obtained by the inventive coupling

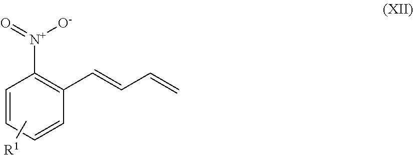

where R¹ is hydrogen, halogen, —CR′(CF₃)₂ where R′ is selected from H, F and O—C₁₋₄-alkyl and is preferably hydrogen, and the R¹ substituent is preferably in the meta or para position, more preferably in the 4 position (para to the NO₂ group) of the aromatic,
may be cyclopropanated to at least one of the compounds (VII) to (IX)

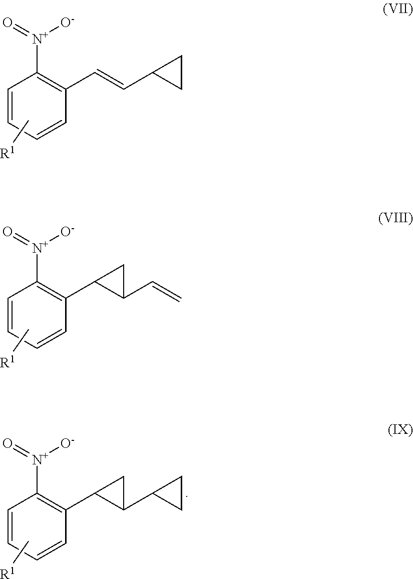

According to the invention, the cyclopropanation is effected by Simmons-Smith reaction with dihalomethane and zinc and/or copper or diethylzinc. The reaction conditions of the cyclopropanation are known to those skilled in the art and have been described before in the prior art, for example, in Org. React. 1973, 20, p. 1-131.

Alternatively, the cyclopropanation can also be effected by carbene addition with diazomethane.

The compounds of the general formulae (VII), (VIII) and (IX) are of significance more particularly as intermediates for active agrochemical ingredients, as described in WO-A-03/074491.

### WORKING EXAMPLES

### 1-[3,3-Dimethylbut-1-en-1-yl]-2-nitrobenzene

To a solution of 6 g (38 mmol) of 2-chloronitrobenzene in 60 ml of DMF are added, under argon, 0.43 g (3.8 mmol) of diazabicyclo(2.2.2)octane, 6.14 g (19 mmol) of tetra-n-butylammonium bromide, 427 mg of palladium(II) acetate, 5.263 g (38 mmol) of potash and 12.8 g (152.3 mmol) of 3,3-dimethylbut-1-ene. The mixture is stirred in an autoclave at nitrogen pressure 5 bar at 130° C. for 20 hours. The mixture is subsequently filtered with suction through Celite, and the filtrate is concentrated under reduced pressure, taken up in ethyl acetate and washed with water. The organic phase is removed and concentrated by evaporation under reduced pressure. This affords 5.5 g (44% of theory) of 1-[3,3-dimethylbut-1-en-1-yl]-2-nitrobenzene in the form of an oil having a purity (GC-MS) of 63%.

¹H NMR (400 MHz, CDCl₃): 1.14 (s, 9H), 6.23 (d, 1H), 6.78 (d, 1H), 7.34 (t, 1H), 7.52 (t, 1H), 7.57 (d, 1H), 7.88 (d, 1H).

1. A nitrobenzene derivative of the formula (IV) 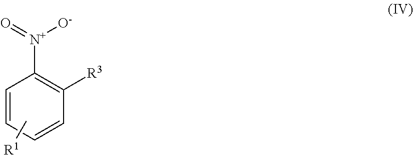
where
R¹ is hydrogen, halogen, or —CR′(CF₃)₂ where R′ is selected from the group consisting of H, F and —O—C₁₋₄-alkyl; and
R³ is —CH═CH-i-propyl, —CH₂CH₂-t-butyl, —CH₂CH₂-i-propyl or 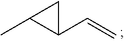
or
R¹ is halogen, or —CR′(CF₃)₂ where R′ is selected from the group consisting of H, F and —O—C₁₋₄-alkyl; and
R³ is —CH═CH-t-butyl,

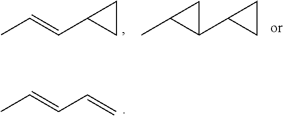

2. A nitrobenzene derivative as claimed in claim 1, of the formula (V) 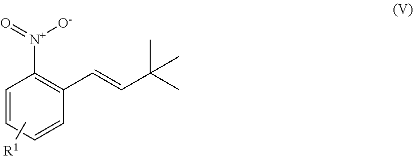
where
R¹ is halogen, or —CR′(CF₃)₂ where R′ is selected from the group consisting of H, F and —O—C₁₋₄-alkyl.

3. A nitrobenzene derivative as claimed in claim 1, of the formula (VI) 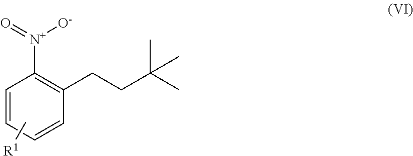
where
R¹ is hydrogen, halogen, or —CR′(CF₃)₂ where R′ is selected from the group consisting of H, F and —O—C₁₋₄-alkyl.

4. A nitrobenzene derivative as claimed in claim 1, of the formula (VII) 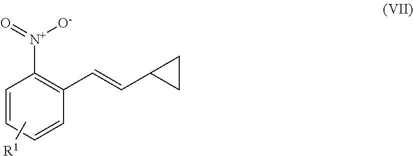
where
R¹ is halogen, or —CR′(CF₃)₂ where R′ is selected from the group consisting of H, F and —O—C₁₋₄-alkyl.

5. A nitrobenzene derivative as claimed in claim 1, of the formula (VIII) 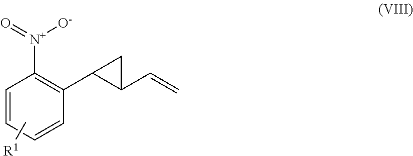
where
R¹ is hydrogen, halogen, or —CR′(CF₃)₂ where R′ is selected from the group consisting of H, F and —O—C₁₋₄-alkyl.

6. A nitrobenzene derivative as claimed in claim 1, of the formula (IX) 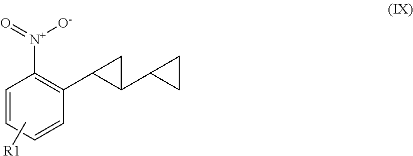
where
R¹ is halogen, or —CR′(CF₃)₂ where R′ is selected from the group consisting of H, F and —O—C₁₋₄-alkyl.

7. A nitrobenzene derivative as claimed in claim 1, of the formula (XII) 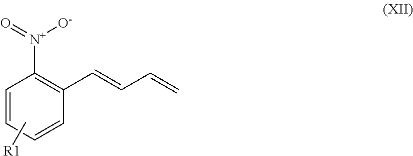
where
is halogen, or —CR′(CF₃)₂ where R′ is selected from the group consisting of H, F and —O—C₁₋₄-alkyl.

8. A process for preparing a nitrobenzene derivative of the formula (I) 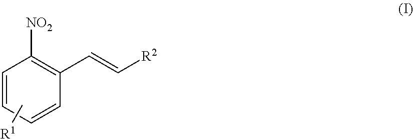
where
R¹ is hydrogen, halogen, or —CR′(CF₃)₂ where R′═H, F or O—C₁₋₄-alkyl, and
R² is i-propyl, cyclopropyl, ethylenyl or t-butyl,
comprising coupling a 2-halonitrobenzene of the formula (II) 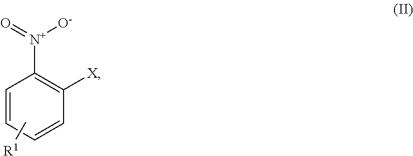
with an alkene of the formula (III)
R²—CH═CH₂ (III)
where
R¹ and R² are defined as above, and
X is a halogen atom,
in the presence of a noble metal catalyst.

9. A process for preparing a compound of the formulae (X) or (XI) 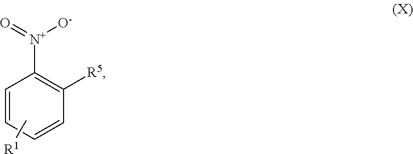 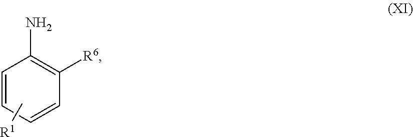
where
R¹ is hydrogen, halogen, or —CR′(CF₃)₂ where R′ is selected from the group consisting of H, F and O—C₁₋₄-alkyl,
R⁵ is —CH₂CH₂-t-butyl, —CH₂CH₂-i-propyl, or —CH₂—CH₂-cyclopropyl, and
R⁶ is —CH₂CH₂-t-butyl, —CH₂CH₂-i-propyl, —CH₂—CH₂-cyclopropyl, —CH═CH-t-butyl,
—CH═CH-i-propyl, 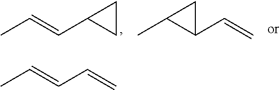
comprising hydrogenating a compound of the formula (I) 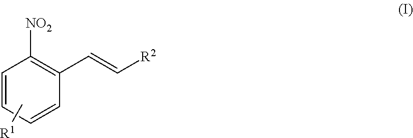
where
R¹ is defined as above, and
R² is i-propyl, cyclopropyl, ethylenyl or t-butyl.

10. A process for preparing a compound of the formula (XII) 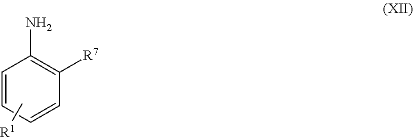
where
R¹ is hydrogen, halogen, or —CR′(CF₃)₂ where R′═H, F or O—C₁₋₄-alkyl, and
R⁷ is —CH₂CH₂-t-butyl,
—CH₂CH₂-i-propyl, 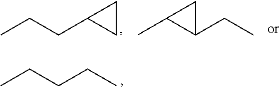
comprising hydrogenating a compound of the formula (XI) 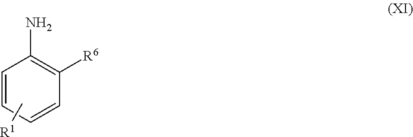
where
R¹ is defined as above, and
R⁶ is —CH═CH-t-butyl,
—CH═CH-i-propyl,

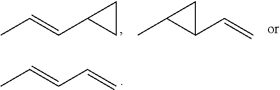

11. A process for preparing a compound of the formulae (VII), (VIII) or (IX) 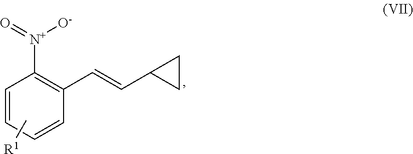 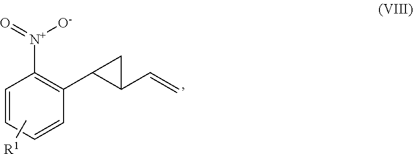 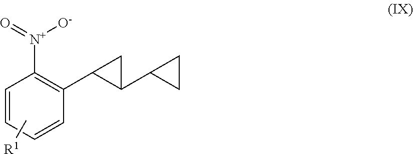
where
is hydrogen, halogen, or —CR′(CF₃)₂ where R′ is selected from the group consisting of H, F and —O—C₁₋₄-alkyl,
comprising cyclopropanating a compound of the formula (XII) 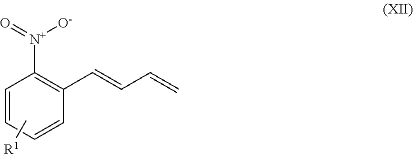
where
R¹ is defined as above.

12. A process for preparing a nitrobenzene derivative of the formula (IX) 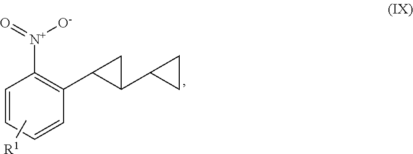
where
R¹ is hydrogen, halogen, or —CR′(CF₃)₂ where R′ is selected from the group consisting of H, F and —O—C₁₋₄-alkyl,
comprising cyclopropanating a nitrobenzene derivative of the formulae (VII) or (VIII) 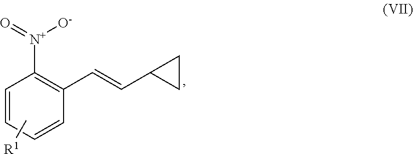 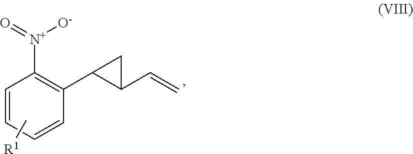
where
R¹ is defined as above.

13. The process as claimed in claim 11, further comprising Simmons-Smith reaction with dihalomethane, zinc and/or copper.

14. The process as claimed in claim 12, further comprising Simmons-Smith reaction with dihalomethane, zinc and/or copper.
